(19) United States
(12) Patent Application Publication
(10) Pub. No.: US 2025/0134592 A1
(43) Pub. Date: May 1, 2025

(54) ANTI-RETROPULSION CATHETER

(71) Applicant: GYRUS ACMI, INC. D/B/A OLYMPUS SURGICAL TECHNOLOGIES AMERICA, Westborough, MA (US)

(72) Inventor: Joey Magno, Dudley, MA (US)

(21) Appl. No.: 18/927,630

(22) Filed: Oct. 25, 2024

Related U.S. Application Data
(60) Provisional application No. 63/592,952, filed on Oct. 25, 2023.

(51) Int. Cl. A61B 18/24; A61B 17/12; A61B 18/00; A61B 18/26; A61M 25/10

(52) U.S. Cl. CPC A61B 18/245; A61B 17/1204; A61B 17/12099; A61B 17/12136; A61B 18/26; A61M 25/10185; A61B 2018/0022; A61B 2018/00517; A61B 2018/00577; A61B 2018/00982

(57) ABSTRACT

Various embodiments disclosed relate to a balloon catheter for use with a ureteroscope. A balloon catheter may include an elongated shaft having a proximal end and a distal end, the elongated shaft shaped for insertion into a patient. A balloon catheter may include a balloon attached at the distal end of the elongated shaft, wherein the balloon is inflatable between a collapsed state and inflated state, and wherein a wall of the balloon comprises a balloon wall material defining plurality of valves that open to allow fluid flow therethrough when the balloon is in the inflated state.

### CLAIM OF PRIORITY

This application claims the benefit of priority to U.S. Provisional Patent Application No. 63/592,952, filed Oct. 25, 2023, which is hereby incorporated by reference herein in its entirety.

### BACKGROUND

Lithotripsy is a common method for fragmenting calculi, or calculi, in the urinary tract, kidneys, and/or bladder. Most lithotripsy devices use ultrasound, laser, or pneumatic energy sources to fragment such calculi. The lithotripter can include a shaft connected to an electrically controlled driver or a pneumatic actuator. The shaft can be inserted into the patient's anatomy to a location near the calculus, and energy can be sent to impact the calculus with the shaft to create a jackhammer or drilling effect on the calculus, thus fragmenting the calculus into smaller elements that are easier to remove. The calculus fragments are then removed.

### SUMMARY OF THE DISCLOSURE

In some aspects, the techniques described herein relate to a system for treatment of a ureteral calculi, the system including: a ureteroscope actuatable for breaking up of the ureteral calculi, the ureteroscope including: a laser fiber actuatable for breaking up the ureteral calculi; and at least one working channel having an opening at a distal end; an anti-retropulsion balloon catheter for use with the ureteroscope, the anti-retropulsion balloon catheter including: an elongated shaft having a proximal end and a distal end, the elongated shaft shaped for insertion into a patient; and a balloon attached at the distal end of the elongated shaft, wherein the balloon is inflatable between a collapsed state and inflated state; and a control unit including at least one tank fluidly connected to the working channel of the ureteroscope.

In some aspects, the techniques described herein relate to a balloon catheter for use during lithotripsy to inhibit retropulsion of one or more calculus fragments, the balloon catheter including: an elongated shaft having a proximal end and a distal end, the elongated shaft shaped for insertion into a patient; a balloon attached at the distal end of the elongated shaft, wherein the balloon is inflatable between a collapsed state and inflated state, and wherein a wall of the balloon includes a balloon wall material defining plurality of valves that open to allow fluid flow therethrough when the balloon is in the inflated state, wherein the plurality of valves are configured to open when the balloon is in the inflated state.

In some aspects, the techniques described herein relate to a method of ablating a calculus, the method including: inserting a balloon catheter, having a balloon in a collapsed state, to locate the balloon distal of the calculus, wherein the balloon includes plurality of valves; expanding the balloon to an inflated state to open the plurality of valves to allow irrigation fluid through the plurality of valves of the balloon catheter; and delivering energy to the calculus for ablating the calculus.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a schematic diagram of an anti-retropulsion balloon catheter in an example.

FIGS. 2A-2C are schematic diagrams of a method of using an anti-retropulsion balloon catheter in an example.

FIGS. 3A-3E are schematic diagrams of a balloon catheter in an example.

FIG. 4 is a flow chart depicting a method of using a balloon catheter in an example.

FIG. 5 illustrates a schematic diagram of an anti-retropulsion device and method in an example.

FIG. 6 illustrates a system diagram in an example.

### DETAILED DESCRIPTION

Discussed herein are devices and methods for use with a lithotripter or other calculus ablation device, such as used for treatment of kidney or ureteral calculi. In an example, a device can help prevent distal movement of calculi and fragments by placing a balloon in the passage distal of the calculus. In another example, a hydrogel can be used to prevent or reduce retropulsion of calculi and fragments. Such a hydrogel can be dispensed through the use of bioabsorabable polymers that are temperature-sensitive. Both methods help address retropulsion and upward migration issues, such as due to lather lithotripsy.

Ureteroscopic lithotripsy can be used for treatment of calculi, such as calculi that fail to respond to medical expulsive therapy (MET) or shockwave lithotripsy (SWL). Ureteroscopes can be used in conjunction with calculi retrieval devices and allow for ureteroscopic calculi extraction. However, during ureteroscopic lithotripsy, there is a possibility of calculi retropulsion (e.g., upward migration or movement) during or after treatment of the calculi. This can cause problems in the calculi removal.

There can be a wide variation in the retropulsion rate during such procedures depending on the kinetic energy of the lithotripter, the ureteric calculi level, and other factors. For example, proximal calculi can have a higher rate of migration than those that are distally located.

A laser pulse, whether it is for fragmenting or dusting mode, can affect clinical use by moving the targeted stone away from laser fiber tip, requiring the user to chase the stone and resulting in longer procedures. In an example, the stone in the ureter is identified and once the laser is fired, the stone is pushed away and disappears from the field of view.

In some cases, additional procedures or secondary procedures are used to deal with migrating calculi or fragments, such as flexible ureteroscopy or SWL, which can incur additional cost and time. In some cases, untreated calculi or fragments can incite breeding of new calculus growth. A variety of approaches have been used to prevent calculi retropulsion and migration, many of which include additional devices and procedures.

Discussed herein, an anti-retropulsion balloon catheter can be used with a ureteroscope to help prevent retropulsion of calculi and allow for more effective retrieval of calculi and fragments. In the example of the balloon device, once inflated, the balloon can block distal movement of the calculus or calculus fragments during ablation (e.g., if they are propelled by a laser). The balloon itself can have several “valves” or slits in the balloon material. These slits can, during the balloon's inflated state, open to allow fluid flow therethrough. For example, irrigation with saline can flow through. This can help prevent large pressure differentials and allow for capturing of calculi fragments.

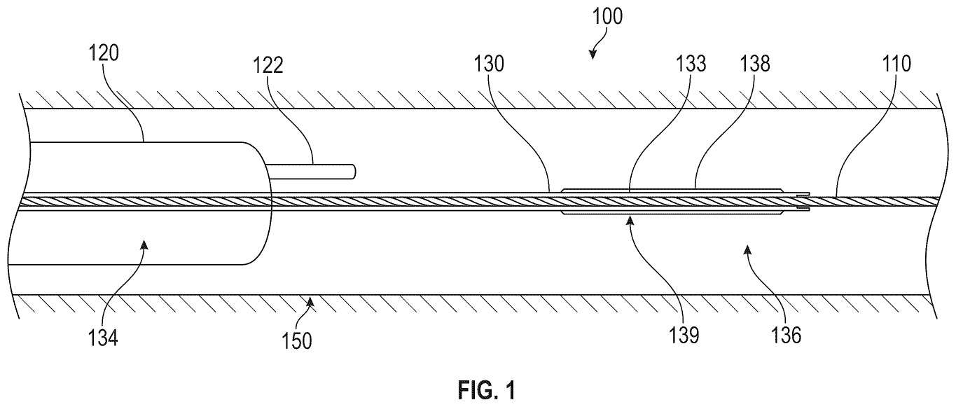

FIG. 1 illustrates a schematic diagram of an anti-retropulsion balloon catheter system 100 in an example. The anti-retropulsion balloon catheter system 100 can include a guidewire 110, a ureteroscope 120, and the anti-retropulsion balloon catheter 130, for use inside the ureter 150. The ureteroscope 120 can include a laser fiber 122. The anti-retropulsion balloon catheter 130 can include an elongated shaft 132 with a proximal end 134 and a distal end 136, a balloon 138 with valves 140.

The anti-retropulsion balloon catheter system 100 can be used in the ureter 150, such as for removal of one or more calculi or other masses in the urology system of the patient. The guidewire 110 can be an elongated wire make of a stiff or semi-stiff material with a diameter smaller than the ureteroscope 120 and the anti-retropulsion balloon catheter 130. The guidewire 110 can be sized and shaped for insertion into the ureter 150. The guidewire 110 can be used to align the ureteroscope 120 and the anti-retropulsion balloon catheter 130 within the ureter 150, such that the ureteroscope 120 and the anti-retropulsion balloon catheter 130 are situated near the target calculus.

The ureteroscope 120 can be a device for fragmentation of a calculi, such as a calculus or other mass, in the ureter. The ureteroscope 120 can be sized and shaped for insertion into a patient's ureter for treatment of the target calculus therein. The ureteroscope 120 can, for example, be a laser-driven device using the laser fiber 122 to deliver laser energy to the calculus for break-up and fragmentation. In some examples, the ureteroscope 120 can be a different type of fragmentation device, such as a sonic, ultrasonic, or mechanical device.

The anti-retropulsion balloon catheter 130 can be inserted along the guidewire 110. The anti-retropulsion balloon catheter 130 can be sized and shaped for insertion into the ureter 150 past the ureteroscope 120.

The shaft 132 can be an elongated shaft sized and shaped for insertion into the ureter 150. The proximal end 134 of the elongated shaft 132 can be attached to a handle or other component for grip and manipulation by an operator. The distal end 136 can host the balloon 138. The anti-retropulsion balloon catheter 130 can be inserted distal of the ureteroscope 120 to help address retropulsion issue due to laser lithotripsy

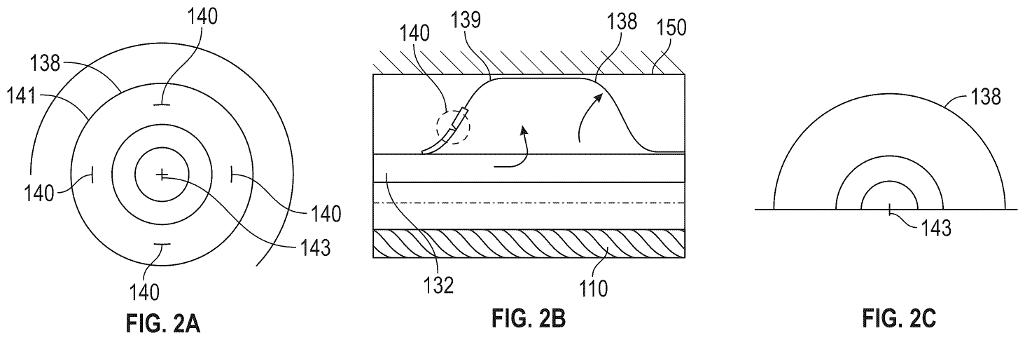

The balloon 138 and valves 140 are shown in more detail in FIGS. 2A-2C. FIG. 2A show a view of the balloon 138 in an inflated or expanded state, looking up the shaft 132 in a distal direction. The valves 140 can be seen in FIG. 2A. FIG. 2B depicts a side view of the balloon 138 in the inflated state along the shaft 132. The proximal portion 139 of the balloon 138 wall can be seen with the valves 140. FIG. 2C depicts a view of the balloon 138 in an inflated state from the distal end 136. The radial center point 143 and the circumference 141 of the balloon 138 can be seen.

The balloon 138 can be made of an inflatable material, such that the balloon 138 is capable of both a collapsed configuration when not inflated, and an expanded configuration when inflated. The balloon 138 can include a wall made of an expandable material. The valves 140 can be located on a proximal portion 139 of the balloon. In some cases, the valves 140 can be distributed about a circumference 141 of the proximal portion 139 of the balloon 138.

The balloon 138 can include the valves 140. The valves 140 can be, for example, slits that allow for movement of fluid therethrough. The valves 140 can be configured to open when the balloon 138 is in an expanded state. In some cases, the valves 140 can be configured to open based on fluid pressure, such as at or above a predetermined threshold pressure. The valves 140 can be, for example, slits within the wall of the balloon 138. In some cases, the wall of the balloon 138 can define the balloon 138.

The balloon 138 can be constructed of a flexible material which inflates easily at about less than 15 inches mercury, such as to seal off an inside diameter of the ureter 150. Once the balloon 138 touches the ureter 150 walls it can cause over pressurizing and it can activate the semi-flexible material of the balloon 138 wall with the valves 140 that open at a predetermined or calibrated pressure. The balloon 138 can be configured such that when inflated, the balloon wall seals off the ureter 150.

A number of valves 140 can be located radially all-around the balloon 138, such as at the proximal portion 139, and can be designed with a declining valve slit thickness towards the center. This thickness can enable the cracking pressure that can open the valve 140 slits to release the flow of saline or other irrigation fluid. This can help address differential pressure between the different sides of the calculi being treated.

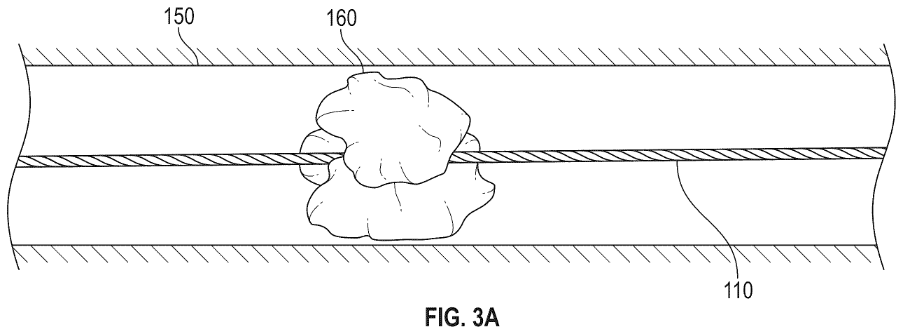

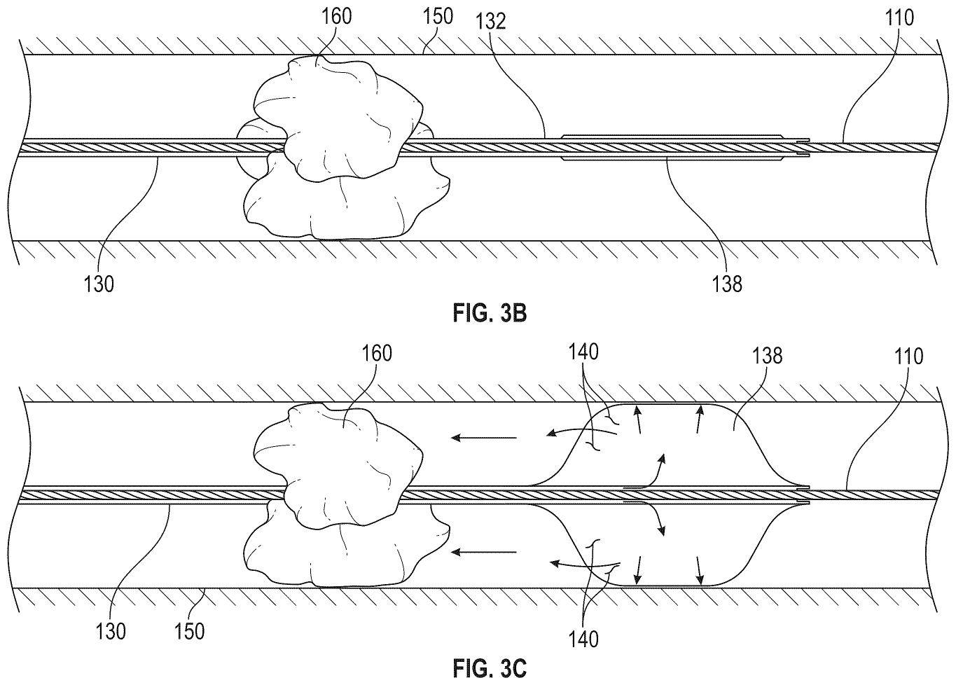

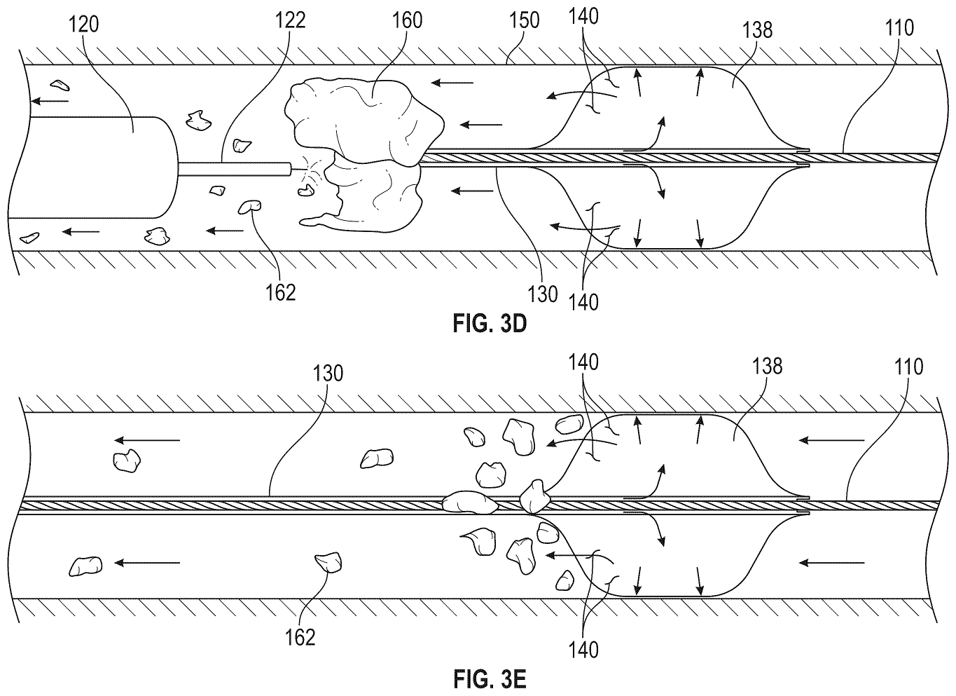

FIGS. 3A-3E are schematic diagrams of a method of using an anti-retropulsion balloon catheter, such as the anti-retropulsion balloon catheter system 100, in an example.

Here, the anti-retropulsion balloon catheter system 100 with the balloon 138 and valves 140 can help address retropulsion issues due to laser lithotripsy by adding the anti-retropulsion balloon catheter 130 to block the area behind the target calculi 160 in the ureter 150. As discussed above, such an anti-retropulsion balloon catheter system 100 can include a guidewire 110, the anti-retropulsion balloon catheter 130 itself, and a flexible ureteroscope 120 with a laser fiber 122.

First, shown in FIG. 3A, the location of the target calculi 160 can be identified, such as by scanning or imaging. For example, a CT scan can be used. Then, the guidewire 110 can be inserted into the patient towards the target calculi 160. The guidewire 110 can be inserted through the calculi 160, such as through the middle of a calculi 160 cluster being targeted. This can be done, for example, with the aid of fluoroscopy.

Next, shown in FIG. 3B, the anti-retropulsion balloon catheter 130 is inserted through the lumen of the ureter 150 along the guidewire 110. This can be done, for example, with the aid of fluoroscopy. In some cases, the distal end 136 of the elongated shaft 132 can include contrasting material for visual aid such as with fluoroscopy.

Shown in FIG. 3C, the balloon 138 on the anti-retropulsion balloon catheter 130 can be activated. The balloon 138 can be inflated. This can be done by introducing irrigation fluid pressure, such as by using saline pressure. Once activated, the balloon 138 can block the far side of the calculi 160. The balloon 138 valves 140 can be situated at the proximal portion 139 of the balloon 138. The valves 140 open once a predetermined or calibrated pressure of the fluid is reached.

Shown in FIG. 3D, the ureteroscope 120 can be introduced with the laser fiber 122. The differential pressure created on both sides of the calculi 160 can prevent the calculi and associated fragments 162 from retropulsion distally and from betting thrown around when the laser fiber 122 is used in dusting or fragmenting the calculi 160. Debris from the calculi 160 can be drained out of the ureter 150 in the proximal direction.

Shown in FIG. 3E, once the calculi 160 are reduced to a drainable size, the ureteroscope 120 can be retracted. The anti-retropulsion balloon catheter 130 can be kept inflated to catch any calculi 160 debris during that retraction.

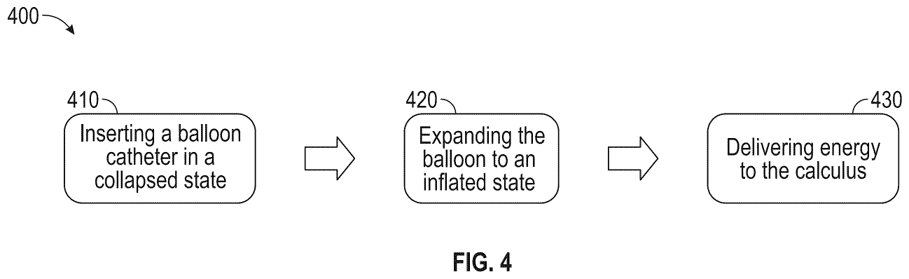

FIG. 4 is a flow chart depicting a method 400 of using a balloon catheter to ablate a calculus in an example. The method 400 can include blocks 410 to 430.

At block 410, a balloon catheter can be inserted into the ureter. The balloon catheter can be in a collapsed state. In some cases, the balloon catheter can be inserted along a guidewire. The balloon catheter can be inserted in a collapsed state. The balloon catheter can include a wall material defining plurality of valves, such as slits therein.

At block 420, the balloon catheter can be expanded to an inflated state to open the plurality of valves, such as to allow irrigation fluid (e.g., saline) to flow through the valves. This can help adjust the pressure differential on either side of the balloon catheter in the ureter. The valves can open at a predetermined or calibrated pressure.

At block 430, energy can be delivered to the calculus such as to ablate, fragment, dust, or otherwise treat the calculus. After ablation, any fragments can be collected or retrieved. The balloon catheter can then be deflated and retracted.

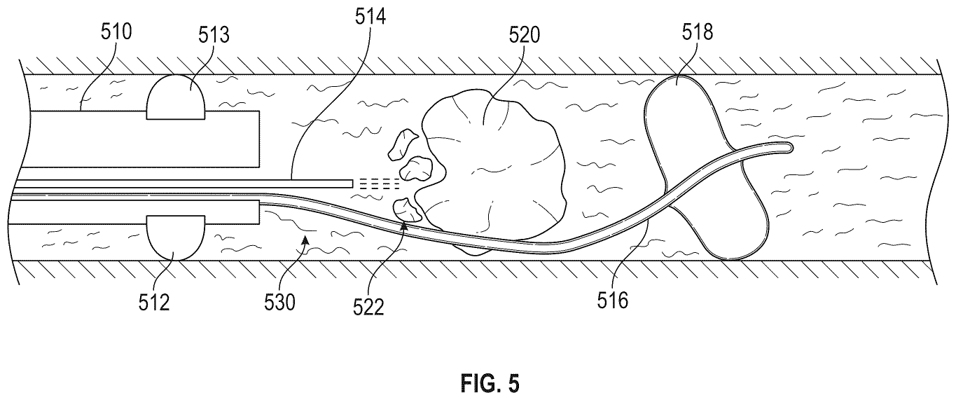

FIG. 5 depicts an example method of addressing retropulsion using a hydrogel. Shown in FIG. 5 are a scope 510 with an integrated scope balloon 513, a proximal occlusion 512, a laser fiber 514, a balloon catheter 516 with a distal occlusion 518, for treatment of a stone 520 with entrapped fragments 522. Used here is a hydrogel 530. The components of the scope 500 can be similar to those discussed above, except where otherwise noted.

Shown in FIG. 5, the target tissue can be identified, such as through a pre-op procedure, for example an MRI or a CT scan. Once identified, the balloon catheter 516, with a deflated balloon, can be advanced to a location distal of the stone 520, such as by fluoroscopy or other imagine techniques. Meanwhile, the scope 510 with the laser fiber 514 can be positioned proximal of the stone 520. The scope 510 can be, for example, a ureteroscope. The laser fiber 514 can be positioned through a working channel of the scope 510.

Here, once the balloon catheter 516 and the scope 510 have been deployed on either side of the hydrogel 530, the distal occlusion 518 can be deployed distal of the hydrogel 530 to help block or seal distal movement of various items, such as the hydrogel 530, entrapped fragments 522, and once applied, the hydrogel 530. Optionally, the scope 510 can include the integrated scope balloon 513 to provide a proximal occlusion 512. This can be deployed for use with the hydrogel as discussed herein.

Once the distal occlusion 518 is deployed, a hydrogel 530 can be sent into the space in and around the stone 520, such as by running or pumping the hydrogel 530 down a working channel of the scope 510. Filling the volume in and around the stone 520, the hydrogel 530 can entrap the stone 520 and entrapped fragments 522 that occur during firing of the laser fiber 514, and prevent the entrapped fragments 522 from retropulsion and migrating all around the patient anatomy both distally and proximally.

The hydrogel 530 can be, for example, a liquid polymer material or a hydrogel material. The hydrogel 530 can, for example, be made of polymers that dissolve in water slightly below ambient temperatures. For example, the hydrogel can be based on PCLA-PEG-PCLA tri-block copolymers with aliphatic end groups. In an example, these polymer sets can include gamma (hydrogel) polymer groups or liquid polymer (LQP) polymer groups. A gamma material can include polymers that dissolve in water slightly below ambient temperatures, and can form hydrogels at pre-set body temperatures. Thus, upon injection into soft tissues, the liquid polymer solution can rapidly form a soft, macroscopic depot, which physically entraps stone fragments. In the example of a liquid polymer material, LQP is a water-free 100% pure liquid polymer without solvents. Such an LQP material, once injected into soft tissue, would rapidly form a soft, macroscopic deposit, which physically entraps stone fragments.

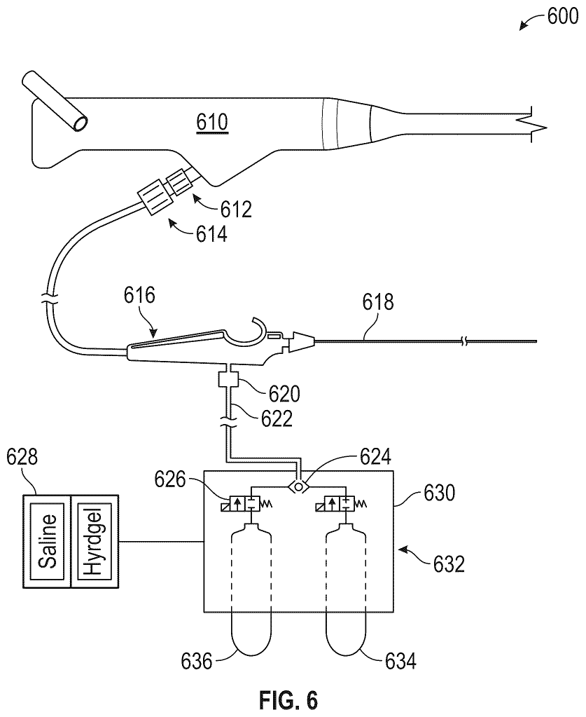

FIG. 6 depicts an example system 600 in which the example set-up of FIG. 5 can

be used. The system 600 can include a ureteroscope 610, a biopsy port 612, an adapter 614, a cannula assembly 616, a laser fiber 618, a luer 620, a saline/hydrogel agent line 622, a shuttle valve 624, 2-way valves 626, a footswitch 628, an air pressure line input 630, a control unit 632, a hydrogel agent tank 634, and a saline container 636.

The ureteroscope 610 can be, for example, similar to the scopes discussed above. The biopsy port 612 and the adapter 614 can connect the ureteroscope 610 to the cannula assembly 616 and the laser fiber 618. The ureteroscope 610 with the laser fiber 618 can be used to treat calculi or other stones. In an example, the device can include the components discussed above with reference to FIG. 5, such as including a balloon catheter allowing for a distal occlusion for insertion of a hydrogel for capture of stone fragments.

The luer 620 and the saline/hydrogel agent line 622 can connect the ureteroscope 610 to the control unit 632, which can be operated to actuate flow of hydrogel or saline through a working channel of the ureteroscope 610 for application in operator.

The control unit 632 of the system 600 can house the hydrogel agent tank 634 and the saline container 636. The shuttle valve 624 and the 2-way valves 626, an air pressure line input 630, can be used to articulate flow of saline and hydrogel from the hydrogel agent tank 634 and the saline container 636 by operation of the footswitch 628. The footswitch 628 can be used to input to the control unit 632 which material should be dispensed, and an electronic controller integrated therein can provide a dispensing operation through the system 600 to the ureteroscope 610. The control unit 632 can optionally be a standalone component, or integrated with the ureteroscope 610. In an example, the system 600 can include such a control unit 632 that further includes a power or light source to the ureteroscope 610 and/or a laser console for lithotripsy procedures.

### Various Notes & Examples

In some aspects, the techniques described herein relate to a system for treatment of a ureteral calculi, the system including: a ureteroscope actuatable for breaking up of the ureteral calculi, the ureteroscope including: a laser fiber actuatable for breaking up the ureteral calculi; and at least one working channel having an opening at a distal end; an anti-retropulsion balloon catheter for use with the ureteroscope, the anti-retropulsion balloon catheter including: an elongated shaft having a proximal end and a distal end, the elongated shaft shaped for insertion into a patient; and a balloon attached at the distal end of the elongated shaft, wherein the balloon is inflatable between a collapsed state and inflated state; and a control unit including at least one tank fluidly connected to the working channel of the ureteroscope.

In some aspects, the techniques described herein relate to a system, wherein a wall of the balloon includes a balloon wall material defining plurality of valves that open to allow fluid flow therethrough when the balloon is in the inflated state.

In some aspects, the techniques described herein relate to a system, wherein the at least one tank includes a hydrogel precursor.

In some aspects, the techniques described herein relate to a system, wherein the hydrogel precursor includes water dissolvable polymers or a liquid polymer actuatable to produce a hydrogel when injected into soft tissue.

In some aspects, the techniques described herein relate to a system, wherein the control unit is actuatable to provide fluid from the tank to a target site around the ureteral calculi.

In some aspects, the techniques described herein relate to a system, wherein the balloon is configured to produce an occlusion distal of the ureteral calculi when in an inflated state.

In some aspects, the techniques described herein relate to a system, further including a second balloon integrated with the ureteroscope.

In some aspects, the techniques described herein relate to a system, wherein the second balloon is actuatable to provide an occlusion proximal of the ureteral calculi.

In some aspects, the techniques described herein relate to a system, further including a footswitch configured to actuate flow of a hydrogel precursor from the tank to the ureteral calculi.

In some aspects, the techniques described herein relate to a balloon catheter for use during lithotripsy to inhibit retropulsion of one or more calculus fragments, the balloon catheter including: an elongated shaft having a proximal end and a distal end, the elongated shaft shaped for insertion into a patient; a balloon attached at the distal end of the elongated shaft, wherein the balloon is inflatable between a collapsed state and inflated state, and wherein a wall of the balloon includes a balloon wall material defining plurality of valves that open to allow fluid flow therethrough when the balloon is in the inflated state, wherein the plurality of valves are configured to open when the balloon is in the inflated state.

In some aspects, the techniques described herein relate to a balloon catheter, wherein an individual one of the plurality of valves includes a slit.

In some aspects, the techniques described herein relate to a balloon catheter, wherein the plurality of valves are configured to open at a threshold pressure.

In some aspects, the techniques described herein relate to a balloon catheter, wherein the plurality of valves are on a proximal portion of the wall of the balloon, and the plurality of valves are distributed about a circumference of the proximal portion of the balloon.

In some aspects, the techniques described herein relate to a balloon catheter, wherein the plurality of valves include varying thicknesses, and the plurality of valves include at least a first valve radially closer to a center point of the balloon and a second valve radially further from the center point of the balloon, wherein the first valve is thicker than the second valve.

In some aspects, the techniques described herein relate to a balloon catheter, wherein the plurality of valves include varying lengths.

In some aspects, the techniques described herein relate to a balloon catheter, wherein the plurality of valves are arranged equally around a center point of the balloon.

In some aspects, the techniques described herein relate to a method of ablating a calculus, the method including: inserting a balloon catheter, having a balloon in a collapsed state, to locate the balloon distal of the calculus, wherein the balloon includes plurality of valves; expanding the balloon to an inflated state to open the plurality of valves to allow irrigation fluid through the plurality of valves of the balloon catheter; and delivering energy to the calculus for ablating the calculus.

In some aspects, the techniques described herein relate to a method, further including retracting the balloon catheter and retrieving one or more fragments of the calculus with the balloon during retraction.

In some aspects, the techniques described herein relate to a method, further including injecting a hydrogel precursor around the calculus to produce a hydrogel for capturing calculus fragments.

In some aspects, the techniques described herein relate to a method, further including producing a distal occlusion with the balloon.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system for treatment of a ureteral calculi, the system comprising:
a ureteroscope actuatable for breaking up of the ureteral calculi, the ureteroscope comprising:
a laser fiber actuatable for breaking up the ureteral calculi; and
at least one working channel having an opening at a distal end;
an anti-retropulsion balloon catheter for use with the ureteroscope, the anti-retropulsion balloon catheter comprising:
an elongated shaft having a proximal end and a distal end, the elongated shaft shaped for insertion into a patient; and
a balloon attached at the distal end of the elongated shaft, wherein the balloon is inflatable between a collapsed state and an inflated state; and
a control unit comprising at least one tank fluidly connected to the at least one working channel of the ureteroscope.

2. The system of claim 1, wherein a wall of the balloon comprises a balloon wall material defining plurality of valves that open to allow fluid flow therethrough when the balloon is in the inflated state.

3. The system of claim 1, wherein the at least one tank comprises a hydrogel precursor.

4. The system of claim 3, wherein the hydrogel precursor comprises water dissolvable polymers or a liquid polymer actuatable to produce a hydrogel when injected into soft tissue.

5. The system of claim 1, wherein the control unit is actuatable to provide fluid from the at least one tank to a target site around the ureteral calculi.

6. The system of claim 1, wherein the balloon is configured to produce an occlusion distal of the ureteral calculi when in the inflated state.

7. The system of claim 1, further comprising a second balloon integrated with the ureteroscope.

8. The system of claim 7, wherein the second balloon is actuatable to provide an occlusion proximal of the ureteral calculi.

9. The system of claim 1, further comprising a footswitch configured to actuate flow of a hydrogel precursor from the at least one tank to the ureteral calculi.

10. A balloon catheter for use during lithotripsy to inhibit retropulsion of one or more calculus fragments, the balloon catheter comprising:
an elongated shaft having a proximal end and a distal end, the elongated shaft shaped for insertion into a patient;
a balloon attached at the distal end of the elongated shaft, wherein the balloon is inflatable between a collapsed state and inflated state, and
wherein a wall of the balloon comprises a balloon wall material defining plurality of valves that open to allow fluid flow therethrough when the balloon is in the inflated state, wherein the plurality of valves are configured to open when the balloon is in the inflated state.

11. The balloon catheter of claim 10, wherein an individual one of the plurality of valves comprises a slit.

12. The balloon catheter of claim 10, wherein the plurality of valves are configured to open at a threshold pressure.

13. The balloon catheter of claim 10, wherein the plurality of valves are on a proximal portion of the wall of the balloon, and the plurality of valves are distributed about a circumference of the proximal portion of the balloon.

14. The balloon catheter of claim 10, wherein the plurality of valves comprise varying thicknesses, and the plurality of valves comprise at least a first valve radially closer to a center point of the balloon and a second valve radially further from the center point of the balloon, wherein the first valve is thicker than the second valve.

15. The balloon catheter of claim 10, wherein the plurality of valves comprise varying lengths.

16. The balloon catheter of claim 10, wherein the plurality of valves are equally spaced around a center point of the balloon.

17. A method of ablating a calculus, the method comprising:
inserting a balloon catheter, having a balloon in a collapsed state, to locate the balloon distal of the calculus, wherein the balloon includes plurality of valves;
expanding the balloon to an inflated state to open the plurality of valves to allow irrigation fluid through the plurality of valves of the balloon catheter; and
delivering energy to the calculus for ablating the calculus.

18. The method of claim 17, further comprising retracting the balloon catheter and retrieving one or more fragments of the calculus with the balloon during retraction.

19. The method of claim 17, further comprising injecting a hydrogel precursor around the calculus to produce a hydrogel for capturing calculus fragments.

20. The method of claim 17, further comprising producing a distal occlusion with the balloon.
